(19) United States
(12) Patent Application Publication
(10) Pub. No.: US 2022/0338730 A1
(43) Pub. Date: Oct. 27, 2022

(54) DEVICE AND METHOD FOR DETECTING TEAR FILM BREAKUP

(71) Applicant: E-SWIN DEVELOPPEMENT, HOUDAN (FR)

(72) Inventors: Yves-Vincent Brottier, HOUDAN (FR); Arnaud Obin, HOUDAN (FR); Nelson Perrin, HOUDAN (FR)

(21) Appl. No.: 17/753,718

(22) PCT Filed: Sep. 11, 2020
(86) PCT No.: PCT/FR2020/051581

§ 371 Date: Mar. 11, 2022

(30) Foreign Application Priority Data
Sep. 13, 2019 (FR) 19 10129

(51) Int. Cl. A61B 3/10

(52) U.S. Cl. CPC A61B 3/101

(57) ABSTRACT

Device and method for detecting tear film breakup, including a backlit translucent plate having a test chart positioned in front of at least one eye of a patient and having a pattern that reflects on the patient's eye, and at least one digital photographic camera connected to a computing system provided with means for processing and analysing images, a lens of the camera pointing towards the patient's eye in order to photograph a reflection of the test chart pattern on the patient's eye, wherein the test chart has a series of lines in the form of alternating horizontal or vertical transparent and opaque lines that form the pattern and at least one opening facing the camera lens.

### CROSS-REFERENCE TO RELATED APPLICATIONS

This application is a National Stage of International Application No. PCT/FR2020/051581, having an International Filing Date of 11 Sep. 2020, which designated the United States of America, and which International Application was published under PCT Article 21(2) as WO Publication No. 2021/048511 A1, which claims priority from and the benefit of French Patent Application No. 1910129, filed on 13 Sep. 2019, the disclosures of which are incorporated herein by reference in their entireties.

### BACKGROUND

### Field

The invention relates to the field of detection of dry eye syndrome.

### Brief Decription of Related Developments

Tests for evaluating dry eye syndrome comprise a test in which practitioners use fluorescein and observe changes in the patient's tear film directly. This so-called invasive test generally measures a time taken for a first tear-film break-up to appear.

A non-invasive test, which measures a non-invasive break-up time, also exists. This test assesses the quality of the tear film, allows the time between an eyelid blink and break-up of the tear film to be determined, and uses reflection from the cornea of a test chart provided with a pattern made up of concentric circles and of radial lines.

When the film breaks up, the lines and/or circles of the reflected pattern become deformed or regions of the reflected pattern go missing.

Images of the reflected pattern are acquired in sequences by a camera and processed by a computerized processing system that analyzes the variation in the pattern over time with a view to detecting deformations of the pattern and to deducing therefrom the time of appearance of film break-up regions.

Currently available devices often use patterns made up of concentric circles and of radial lines. This causes problems of a number of kinds:

On account of the shape of the test charts, and of the coverage of the cornea by disc sectors, spatial resolution is intrinsically worse at the edges of the field than at its center.

At the center of the field, where the radial lines converge, it is difficult to determine the deformation of a pattern.

To increase spatial resolution the density of the pattern of the test chart must be increased. Furthermore, algorithms for detecting deformations of circles, which require the most probable circles to be found in the image, whether a point in the image belongs to a probable circle to be evaluated and the distance of a given point to the circle to which it is supposed to belong to be computed, are complex and the processing and computing time taken to detect break-up regions on the basis of the image is substantial.

It will therefore be clear that: on the one hand, devices employing the conventional pattern of concentric circles and radial lines suffer from a lack of uniformity in spatial resolution, and, on the other hand, increasing resolution at the edge of the field adversely affects computing time.

Due to the low resolution of this method, the measurement may result in inaccurate and overestimated break-up times.

Thus there is real uncertainty in whether or not tear-film break-up has occurred and, furthermore, algorithms for detecting deformations of circles, which require the most probable circles to be found in the image, whether a point in the image belongs to a probable circle to be evaluated and the distance of a given point to the circle to which it is supposed to belong to be computed, are complex and the processing and computing time taken to detect break-up regions on the basis of the image is substantial. In addition, as seen above, measurement accuracy is low and machines using this principle are not sensitive enough to measure small break-ups, especially because resolution decreases with distance from the center of the pattern composed of circles and radial lines. However, practitioners need a reliable and reproducible measurement to carry out diagnosis.

### SUMMARY

The invention improves the situation and firstly makes provision to use a device comprising a pattern made up of light and dark lines, and to observe the reflection of this pattern from the cornea.

To do this, the invention proposes, according to one aspect, a device for detecting one or more break-ups of a tear film, comprising a backlit translucent plate equipped with a test chart that is positioned in front of at least one eye of a patient and that is provided with a pattern that is reflected from the eye of the patient, and at least one digital photographic camera connected to a computing system that is internal or external to the measuring device and that is provided with means for processing and analyzing images, an objective of the camera pointing toward the patient's eye in order to photograph a reflection of the pattern of the test chart from the patient's eye, wherein the test chart is provided with a series of lines taking the form of an alternation of horizontal or vertical transparent lines and opaque lines making up said pattern and at least one aperture facing the objective of the camera and wherein, the transparent lines forming, on the backlit translucent plate, light lines that are reflected from the eye of the patient whereas the opaque lines are reflected as dark lines, the means for processing and analyzing images are configured to detect deformations of said light or dark lines of the pattern of the test chart reflected from the patient's eye and to identify tear-film break-ups revealed by these deformations.

The device of the present invention, which device is based on detection of light or dark lines in an alternation of light and dark lines, avoids the problems of devices employing test charts with circular patterns and allows fine detection of film break-ups, even though, at first sight, it would appear to be inapposite to the eye, which has a spherical curvature.

Furthermore, because the resolution of the pattern remains uniform over the entire region measured, the present invention allows a series of measurements tracing the progress of the growth of tear-film break-ups to be carried out.

The device features described in the following paragraphs may, optionally, be implemented. They may be implemented independently of one another or in combination with one another:

The lines of the series of lines are preferably parallel lines.

The width of the lines advantageously increases from a median line of the test chart toward the edges of the test chart.

The test chart is provided with a cylindrical curvature generated using a vertical generatrix.

The test chart is advantageously of sufficient size to produce an image on most of the cornea of the patient's eye or eyes; for example, it is designed to fill most of the human field of view.

The means for processing and analyzing images may advantageously comprise means for converting the image to grayscale.

The means for processing and analyzing images may advantageously comprise anisotropic band-pass filtering means.

The means for processing and analyzing images may advantageously comprise means for analyzing the image in successions of pixels perpendicular to the direction of the lines, means for searching for bright or dark segments in said successions of pixels, and means for quantifying the size of said bright or dark segments and for removing segments the size of which is incompatible with an image of the lines of the pattern.

The means for processing and analyzing images may advantageously comprise means for marking/cataloging bright or dark segment of objects, which means are suitable for reconstructing first objects corresponding to lengths of bright or dark lines of the pattern and for removing second objects of shape incompatible with said light or dark lines.

The means for processing and analyzing images may advantageously comprise computing means for joining lengths of light or dark lines on the same axis, means for computing a polynomial regression on the light- or dark-line data so as to compute RMS curves representative of edges of said lines and computing means for detecting regions of break-up of the tear film for image points of the line edges the distance of which to said curve is larger than a given tolerance value.

According to another aspect of the present patent application, which may be used independently of the test chart described above, the device may comprise means for tracking the patient's eye or eyes based on iris recognition and tracking.

These means here allow the detected tear-film break-ups to be aligned with the analyzed eye but could be used in other contexts in which it is desired to track the iris.

According to one particular aspect of the device of the present patent application, the backlit translucent plate bearing the test chart and the one or more cameras are integrated into an ophthalmic measuring apparatus, such as an ophthalmological structure equipped with a support on which the head of the patient rests, or integrated into a headset worn by the patient.

The present patent application further relates to a method for detecting tear-film break-ups by means of a device such as described above, and which comprises detecting an eyelid blink that delivers a start time, and performing at least one sequence comprising successively capturing images and computing regions of break-up from the start time to the next eyelid blink.

The method features described in the following paragraphs may, optionally, be implemented. They may be implemented independently of one another or in combination with one another:

The method may comprise capturing an image every 0.2 to 0.5 seconds and preferably every 0.3 seconds.

The method advantageously comprises, for each captured image, a succession of processing and analyzing steps comprising
converting the image to grayscale;
filtering the converted image by means of an anisotropic band-pass filter in order to decrease vignetting and to increase the uniformity of the brightness of the image;
searching for bright or dark segments in the image column by column, a step of quantifying the size of said bright or dark segments and a step of removing segments the size of which is incompatible with a correspondence with lines of the pattern;
a step of marking/cataloging bright segments or dark segments of objects, of reconstructing first of said objects corresponding to objects forming lengths of lines of the pattern, and a step of removing second of said objects of shape incompatible with said lines of the pattern;
a step of joining lengths of lines of the same level, and a step for computing a polynomial regression on the line data so as to compute an RMS curve of the line edges;
computing regions of break-up of the tear film by computing the distance of points of line edges to said curve, said regions of break-up corresponding to line edges the distance of which to said curve is larger than a given tolerance value.

In this method, the anisotropic filtering and the polynomial regression are image-processing means that together guarantee rapid image processing and good detection of the defects occasioned by tear-film break-ups.

According to one aspect of the patent application that may be used in another method, the method comprises steps of tracking the patient's eye or eyes by means of an iris-tracking method. These steps here allow the detected regions of break-up of the tear film to be repositioned with respect to the analyzed eye.

The eye tracking steps may comprise:
transforming the image a first time via application of an anisotropic band-pass filter that is applied in the direction of the width of the eye, to produce pairs of rising, dark to light, and falling, light to dark, transitions along a horizontal axis of the eye;
segmenting the image to find the pairs of rising and falling transitions, which form segments that are necessarily representative of bright regions in the image;
filtering the image, which removes light segments from the central region comprising the pattern and top and bottom regions of the image;
taking into account light segments, the other regions of the image no longer being considered in this analysis, and computing a first time an RMS circle of the perimeter of the iris on the basis of the right ends of the light segments on the left of the image and of the left ends of the light segments on the right of the image;
a step of removing points that are too far from the RMS circle; and
as regards the remaining points, a new step of computing an RMS circle (93) to follow the outline of the iris.

This patent application further provides a computer program comprising instructions for implementing the method for detecting break-ups and the eye-tracking method when this program is executed by a processor.

This program may be stored on a computer-readable non-volatile storage medium on which is stored a program for implementing the method when this program is executed by a processor.

The patent application lastly proposes a test chart for implementation of the device of the invention, which is produced by means of a transparent polymer film provided with opaque lines that are printed or screen-printed on said film, comprising at least one hole encircled by an opaque frame in a median region of the test chart, said film being designed to be positioned on a backlit translucent plate of the device.

### BRIEF DESCRIPTION OF THE DRAWINGS

Other features, details and advantages of the invention will become apparent on reading the detailed description that follows, and on analyzing the appended figures in which:

FIG. 1 is a plan face-on view of a test chart provided with a pattern of the invention;

FIG. 2 is a schematic view of a device of the present patent application;

FIG. 3 shows a first example of measurement apparatus usable in the context of the present patent application;

FIG. 4 shows an image of a patient's eye following a first treatment step;

FIG. 5A, FIG. 5B, FIG. 5C, FIG. 5D and FIG. 5E show various stages of processing the image of the eye of FIG. 4;

FIG. 6A and FIG. 6B show details of FIG. 5E;

FIG. 7 shows an image of the eye of FIG. 4 after image analysis;

FIG. 8 shows an image of an eye of a patient looking toward a camera;

FIG. 9A, FIG. 9B and FIG. 9C show steps of determining the position of the iris of the eye of FIG. 8 according to one aspect of the patent application;

FIG. 10 shows an image of an eye of a patient whose gaze has deviated from a camera;

FIG. 11A, FIG. 11 B and FIG. 11C show steps of determining the position of the iris of the eye of FIG. 10 according to one aspect of the patent application;

FIG. 12 schematically shows a method for detecting regions of tear-film break-up;

FIG. 13 schematically shows a method for repositioning regions of tear-film break-up in a frame of reference tied to the eye.

### DETAILED DESCRIPTION

The drawings and the description below describe one or more examples of embodiment that will therefore possibly not only be used to better understand the subjects of the present patent application, but also contribute to its definition, where appropriate.

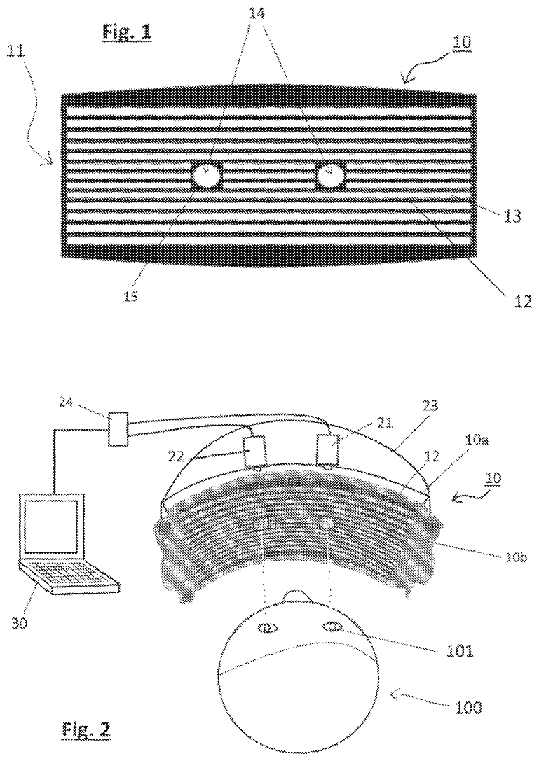

The method and the device for detecting tear-film break-ups according to the present patent application use a test chart 10 (shown in FIG. 1) that is produced from a transparent polymer film comprising a pattern 11 made up of alternating straight and parallel lines 12, 13. The pattern comprises opaque lines 12, black lines for example, separated by transparent lines 13 that let pass the light of a light source, which passes through a translucent carrier behind the pattern, so as to produce light lines, the light lines of the pattern being reflected from the eye or eyes of a patient.

In this example, the pattern 11 of the test chart 10 comprises twelve opaque lines 12, excluding the top and bottom borders of the test chart. These opaque lines are separated by transparent lines 13 and centered on a translucent median line. The test chart may be mounted on a plastic frame in order to make it easier to handle.

By convention, an axis parallel to an axis passing through the patient's eyes will be called the horizontal axis, and the axis perpendicular to this axis will be called the vertical axis; in the illustrated example, the lines of the pattern are horizontal.

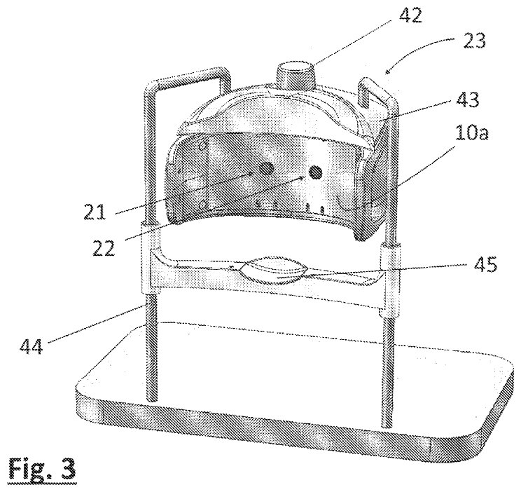

As shown in FIG. 2, the test chart 10 on its plastic carrier 10b is positioned on a translucent carrier 10a itself drilled with holes for passage of camera objectives, and the device uses a diffuse light source 23 behind the carrier of the pattern to illuminate the pattern. The reflection of the pattern is observed by one or two digital cameras and, to observe both eyes, two digital cameras 21, 22 are provided.

The diffuse light source 23 may be produced by means of an integrating box or sphere or similarly to an LCD backlight for example.

The test chart is provided with two empty regions 14 centered in opaque frames 15 on a horizontal median line. The empty regions are separated by a distance corresponding to an average interocular distance, as shown in FIG. 1. Returning to FIG. 2, the cameras are positioned behind the empty regions and in front of the eyes 101 of the patient 100. The objectives of the cameras film or photograph the patient's eyes through the empty regions. The empty regions may be replaced by transparent substrate regions of the test chart if the optical properties of the substrate of the test chart and its level of cleanliness are compatible with image formation (transparent and non-scattering).

The cameras are for example CMOS cameras with 1/4″ sensors. According to the non-limiting example shown, the transparent regions are circular holes of a diameter suitable for the objectives of the cameras. For cameras with optics of focal length of the order of 4 mm, holes of the order of 14 mm are provided and the opaque frames are opaque squares of the order of 16 mm×16 mm. The aim of these frames is to precisely terminate the lines upstream of the holes that receive the objectives of the cameras.

In this example of embodiment, the one or more cameras deliver images with a definition of 1920×1080, which is sufficient to obtain an analysis of film break-up without unduly increasing the computational load on the system.

The video signals or the signals of the cameras are sent to a computerized processing device 30 or computing system that is either internal or external to the measurement device, and, to avoid duplication of this processing device, the video signals of the two cameras are passed, via a multiplexer, on a circuit board 24 of the device, the multiplexer allowing either of the video channels to be sent to the processing device 30 as desired.

The cameras film or photograph the image, of the lines of the pattern, reflected from the patient's cornea. As the cornea, to a first approximation, may be considered to be a spherical dioptric interface, it has a high field curvature and the image exhibits substantial distortion. In order to at least partially compensate for this distortion and to ensure, in the captured images, that the width of the lines varies little from the median axis of the pattern to the edge of the image, the pattern of the test chart comprises lines the period of which increases from the central axis of the pattern to the edges of the carrier that are parallel to the lines. For example, a central white line may have a height of about 2.8 mm, the adjacent black lines a height of 2.2 mm, while the last white lines have a height of 3.8 mm and the black lines preceding these white lines have a height of 2.8 mm, the progression being optimized to compensate for the curvature of a standard eye. In the described method, light lines are used to find regions of break-up. The number and width of the light lines may be different from the given examples, but are chosen to obtain a definition sufficient to achieve a meaningful detection of the regions of film break-up given the resolution of the one or more cameras.

In the context of the present patent application, the white lines are reflected by the corneal dioptric interface and stand out whereas they are backscattered from the bulbar conjunctiva of the eye and the iris, the alternation of dark lines and light lines in contrast forming a continuous background the brightness of which depends on the ratio between the transparent areas of the test chart and its total area.

In FIG. 2, the test chart, seen from above, is curved about a vertical axis to form a portion of a cylinder and to follow the curvature of the patient's head 100 so that the image of the test chart covers most of the cornea.

The reasons therefor are:
a. Optical conjugation:
The camera sees the reflection of the test chart from the cornea, which is considered, to a first approximation, to be a spherical mirror with a radius of about 8 mm, or 4 mm focal length. Given this short focal length, the test chart (the object in the optical conjugation) must be large for the size of the image reflected by the cornea to be large enough, i.e. comparable in size to the outside diameter of the iris. This is what determines the size of the mask.
b. Photometry:
For the test chart to be visible, light rays from the extreme edges of the test chart must enter the pupil of the objective. Since the cornea is a mirror of high curvature, it is necessary, on the field edge, for the light rays reaching the cornea to be grazing.

This is what justifies the curvature of the test chart.

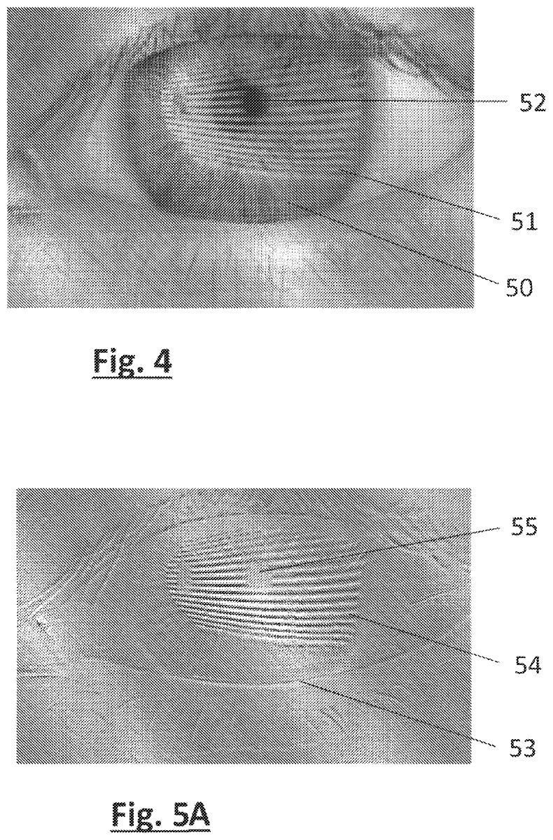

In FIG. 3, the measuring device is mounted on an ophthalmological structure with a chin and forehead rest, the structure comprising uprights 44 and a casing 43 into which are integrated the cameras and that receives the test chart 10 on a frontal curved face in front of which the patient places himself, chin resting on a support 45. With such a structure, the patient's eyes are at a distance from the test chart of about 50 mm for a camera of 4 mm focal length. The measuring device may also be integrated into a headset worn by the patient.

Allowing for variations in the position of the eye with respect to the camera (different interocular distance from one individual to the next) the focal length and the distance are chosen to permit the whole eye to be seen. Next, a window of interest centered on the patient's pupil is chosen, via an action of the operator, who marks the center of the pupil in the image.

So that the lines of the test chart are sharp in the image, the focus is adjusted with a thumbwheel 42.

The object of the measuring method of the present patent application is to detect and measure the growth of regions of eye dryness and of tear-film break-up. The measuring method comprises repeatedly capturing images of one or both eyes of the patient at a repetition rate of the order of 0.2 to 0.5 seconds and in practice of 0.3 seconds, after a blink of the eyes or of the eye.

The method is mainly described in the context of a test chart composed of lines that are horizontal, i.e. that extend along an axis passing through the two pupils of the patient, but is adaptable, especially by means of a 90° rotation of the means for processing series of pixels and of the anisotropic band-pass filter described below. Moreover, the method, which is described in the context of detection of the light lines, is applicable to detection of the dark lines.

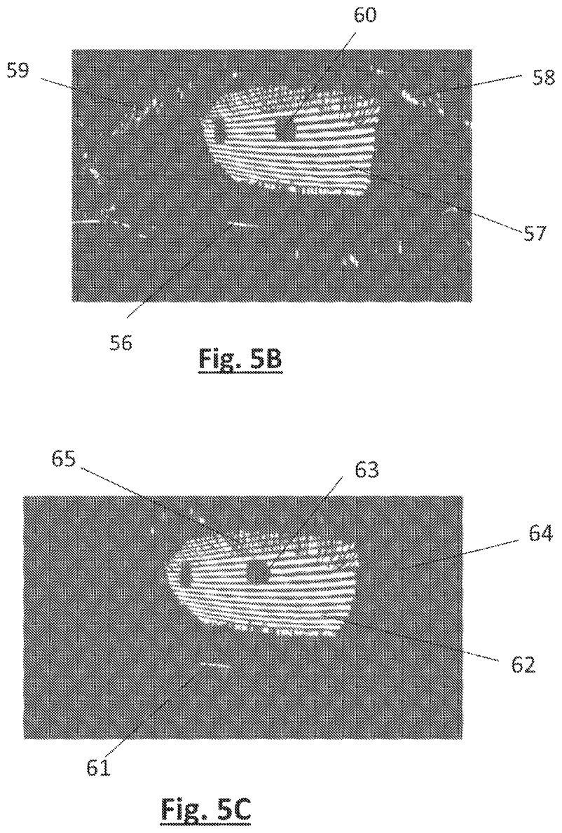

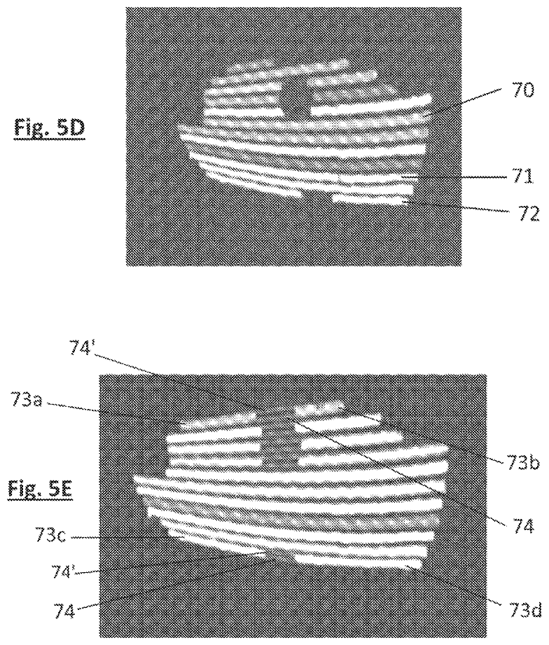

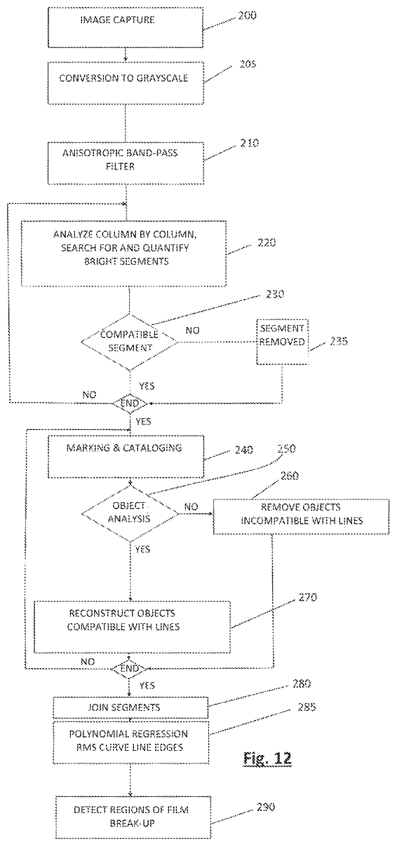

In the computing system 30, the measuring steps comprise image-processing steps that, starting from capture of the original image of the patient's eye, comprise:
converting (step 205 of FIG. 12) the image into grayscale, an example of the result of which is shown in FIG. 4 in which may be seen the image 51 of the pattern on the iris 50 with the image of the frame 52 encircling the objective of the camera. In this image and in the original color image, the shape of the reflected light lines comprises local defects (especially irregular line edges that alone imply deformation of the tear film);
a second step (step 210 of FIG. 12) comprises applying an anisotropic band-pass filter, which filter is applied in a direction perpendicular to the direction of the reflected lines, i.e. either a vertical direction, and therefore to columns of pixels of the image, in the case of a test chart composed of horizontal lines, or of a test chart composed of vertical lines, the image being in the latter case rotated by 90° to obtain light lines oriented in a horizontal direction, and which filter is configured to pass abrupt transitions between grayscale levels and to remove or attenuate modulations of low spatial frequency and of higher spatial frequency in the perpendicular direction. The image output from the filter is shown in FIG. 5A. This filter especially has the advantage of removing vignetting and defects in lighting uniformity. This transformation accentuates the edges of the eyelid 53, the light lines 54 of the pattern of the test chart and preserves the image of the frame 55.

Next, again in the case of light lines oriented in a horizontal direction, the method comprises analyzing 220 the image in columns of pixels, as shown in FIG. 12, to search for bright vertical segments. The resulting image then comprises lines 56, 57, 58, 59 as shown in FIG. 5B. Once this analysis has been carried out, the bright segments are qualified by their size, in steps 230 and 235. This allows segments that are too large or too short, and that clearly do not correspond to segments of lines of the pattern, as for example is the case of segments forming part of the outline of the eyelids, to be removed. FIG. 5C shows, at the end of this step, the image, in which the column segments of the isolated line 61, the lines of the image of the pattern 62, the background 64 and the frame 63 remain. It will be noted that the eyelashes cause a substantial fragmentation of the lines 65 at the top of the image. In the case of a test chart composed of vertical lines, the segments are analyzed and qualified in rows of pixels.

When the segmentation has ended, the processing method comprises an algorithm for marking/cataloging 240, 250, 260 the bright segments to obtain objects representative of bright lengths of lines of the pattern and to reject bright objects not having the desired shape, which are thus considered artifacts. This algorithm firstly joins contiguous column segments to reconstruct horizontal lengths. The result of this marking/cataloging is shown in FIG. 5D, in which each line found has been assigned a color here represented in grayscale. This cataloging allows complete lines 70 or isolated lengths 71, 72 to be created.

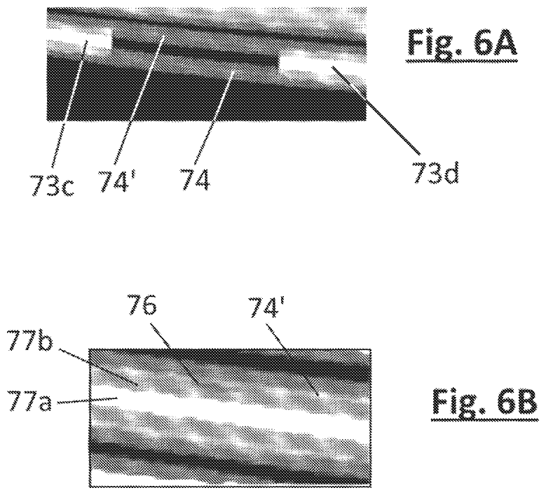

In a subsequent step, the lengths of bright lines of same level (for example of similar width and altitude) in the image are joined 280, then a polynomial regression 285 using a polynomial of order higher than two is applied so as to compute an RMS curve of the shape of the line edges. This step is shown in FIG. 5E. In this figure it will especially be noted that lengths 73a, 73b of the lines fragmented by the image of the frame encircling the objective of the camera, and lengths 73c, 73d of the bottom line, have been joined by top 74 and bottom 74′ RMS curves. The enlargement of FIG. 6A allows the RMS curves 74, 74′ between the lengths 73c, 73d at the bottom of the image to be seen more clearly. Next, sites or regions of film break-up are detected 290, in places where the edges of the lines comprise measurement points that deviate from the shape given by the polynomial, using two criteria:
the deviation from the polynomial is larger than a threshold,
this exceedance of the threshold exists in a plurality of contiguous columns.

This is for example shown in FIG. 6B in the region 76 in which the line edge 77b of the line 77a does not follow the curve 74′.

As seen above, the method may be based on processing of the dark lines. Grouping pairs of transitions (rising and falling transitions in the case of light lines) allows the consistency of the width of the obtained segment to be checked and segments that are too wide or too narrow to form part of the image of the test chart to be rejected. Once the lengths have been determined, the polynomial regression is performed on each side of the length: one polynomial for the rising transitions, one polynomial for the falling transitions. The method of the invention therefore makes it possible to equally well target dark segments and dark lines, the same polynomial regressions and the same final result being obtained.

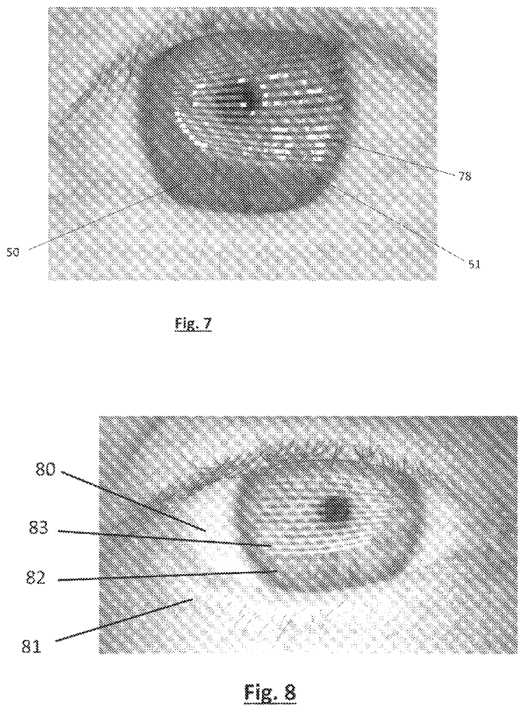

The result of the measurements is a map of sites of tear-film break-up, such as shown in FIG. 7, in which the map of the points 78 of break-up has been positioned on the initial color image of the eye, which image is here shown in grayscale.

As seen above, images are captured about every 0.3 seconds. A start time is defined by an eyelid blink, and repeating the measurement for each image over a time period allows a map of break-ups as a function of time to be constructed.

One problem to be considered is that the patient's gaze may change direction during the image-acquisition period.

Since the camera observes reflections of the pattern from the cornea, which to a first approximation behaves like a spherical dioptric interface, the position of the image of the pattern remains almost invariant in the image delivered by the camera, whereas the position of the iris changes if the patient turns his eyes. Thus, a given point of the image of the test chart is not tied to a fixed point of the cornea, but rather to a point dependent on the direction of the gaze. This implies that the measurement must be referenced with respect to the position of the observed eye and not with respect to the image of the pattern.

To do this, the position of the eye in each image has to be tracked. It is preferable to follow the outline of the iris of the eye because it contrasts highly with the bulbar conjunctiva, which is light in color and from which there is no reflection of the test chart, the pupil being trickier to follow due to the reflection of the test chart, which complicates analysis of the image.

The following method may be implemented in the context of the present patent application or independently to perform other measurements on the eyes. This method is moreover independent of the orientation of the lines of the test chart.

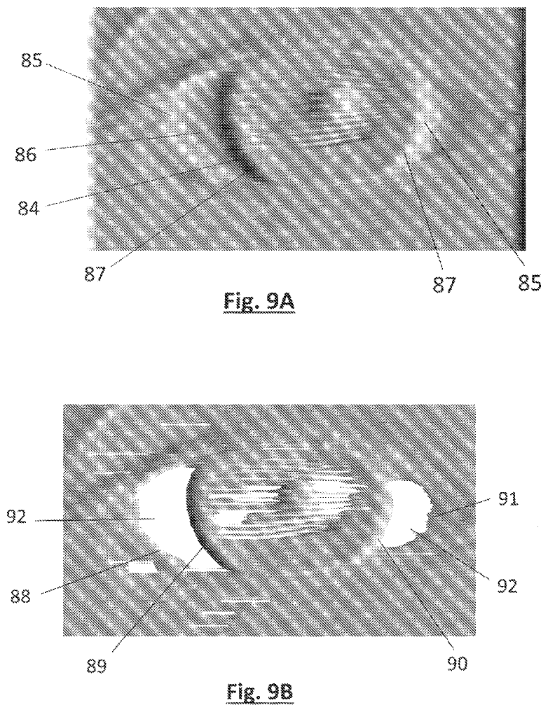

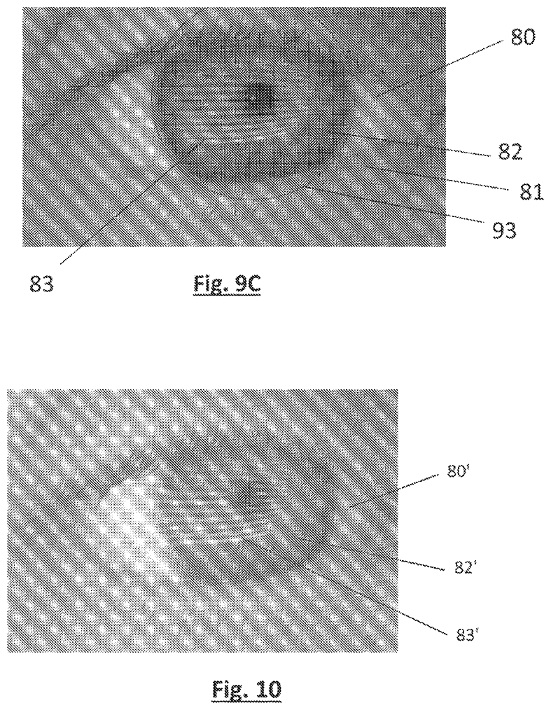

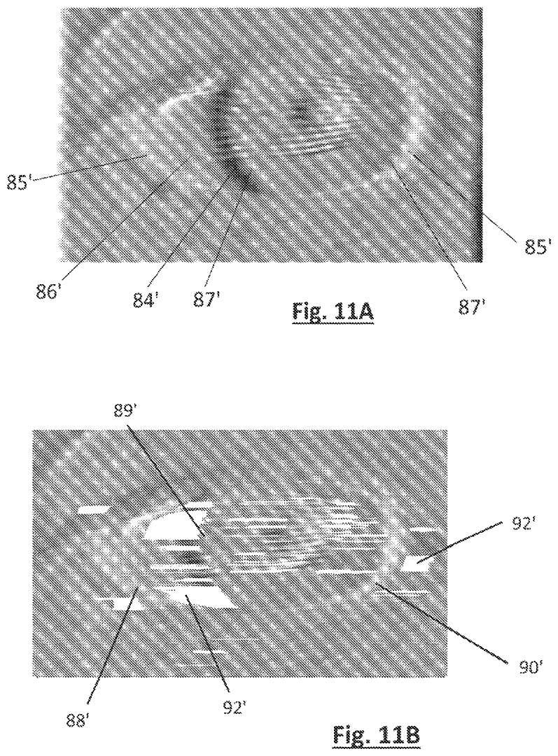

FIGS. 8, 9A, 9B and 9C correspond to processing operations carried out on images of an eye of a patient looking at the camera, and FIGS. 10, 11A, 11B and 110 correspond to processing operations carried out on images of an eye of a patient whose gaze has deviated from a camera.

In FIG. 8, the eye 80 is looking straight ahead, and the image of the pattern 83 is centered with respect to the iris 82, which itself is centered with respect to the eyelid 81.

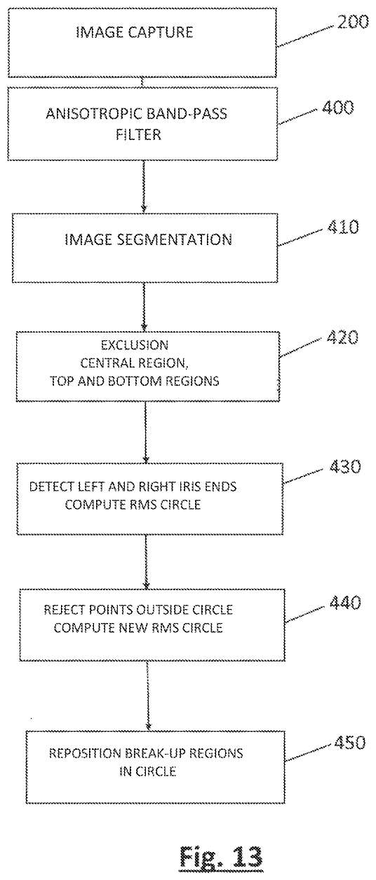

The image-processing method employed to find the position of the iris is schematically shown in FIG. 13. It comprises a first transformation of the image, achieved via application of an anisotropic band-pass filter 400, which is applied horizontally so as to detect transitions in brightness along a horizontal axis. In this operation it is sought to distinguish between falling transitions (light to dark) and rising transitions (dark to light) and, for the sake of legibility, light-to-dark transitions (falling transitions) have been represented by the dark crescent 84 in the grayscale image in FIG. 9A and dark-to-light transitions (rising transitions) have been represented by the light crescent 85 in FIG. 9A. Portions of the image in which there are no significant transitions, such as the crescent 86 for example, become an average gray, and the outline of the iris is manifested by a ring portion 87 that is located, on the left-hand side, next to a light-to-dark transition and, on the other side of the eye, next to a dark-to-light transition.

Returning to FIG. 13, a second operation consists in segmenting the image 410 with a view to finding pairs of rising and falling transitions (84, 85 in FIG. 9A for example) forming the borders of the bright regions in the image, especially around the bulbar conjunctiva. These pairs of transitions, indicated by the limits 88, 89 and 90, 91 in FIG. 9B, frame regions 92 potentially defining the bulbar conjunctiva.

After this transformation, the method comprises, as shown in FIG. 13, filtering the image 420, which removes the central region comprising the pattern and the top and bottom regions of the image. On the basis of the remaining parts, an RMS circle is computed for the perimeter of the iris, from the right ends of the left-hand segments of the image and from the left ends of the right-hand segments of the image 430. For this computation, the points too far from the RMS circle, which correspond to imperfections especially caused by the eyelashes or the eyelid, are rejected in step 440 and, as regards the remaining points, a new RMS circle is computed to follow the outline of the iris, this circle 93 being shown in FIG. 9C on the original image of the eye.

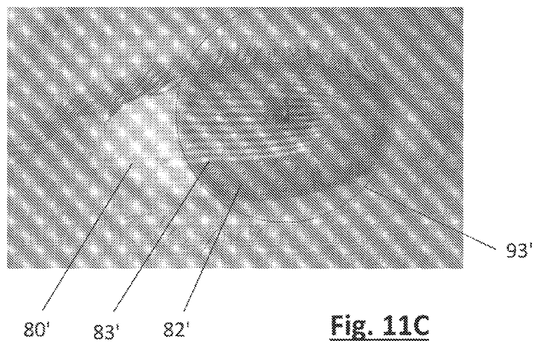

FIG. 10 shows an eye 80′ looking sideways and the iris 82′ of which is offset with respect to the pattern 83′. For this position of the eye, the transitions 84′, 85′ around dark regions 86′, 87′ corresponding to uniform colors are offset laterally in FIG. 11A, though in FIG. 11 B it may be seen that the circular arcs 89′ and 90′ corresponding to the edge of the iris remain detectable. Application of the tracking method once again leads to recreation of the RMS circle 93′, which will be repositioned in the original image as shown in FIG. 110. The detected tear-film break-ups are then relocated, in step 450, depending on the position of the circle defining the outline of the iris. This makes it possible to anchor the break-ups of the tear film to the outline of the eye, rather than to the image.

This sequence is carried out for each image, preferably after the analysis of the line pattern described above.

As stated above, this method is here applied to reposition the regions of break-up, but it may also be used for other types of detection and methods that require the position of an eye to be tracked.

According to one aspect of the patent application, the device may comprise a manual trigger that arms the device, the sequence of image captures then being trigged on the occurrence of an event such as a series of two blinks of the eyelids of the patient. To do this, the system comprises a method for recognizing a blink of the eyelids, which allows the measurement sequence to be started automatically. Likewise, the system may stop the sequence of measurements automatically, on detection of a subsequent blink of the eyelids, or stop the sequence automatically after a time delay, of 15 seconds for example.

The sequence of image captures may comprise from 30 to 50 images for example, and in the case of a sequence of image captures of 15 seconds length with images captured every 0.3 seconds the sequence comprises 45 images. The images may be analyzed after the sequence of image captures and, because of the chosen solution, i.e. the choice to work with a pattern made up of lines, the processing time remains low, for example 15 seconds with a standard computer.

Once measurement is over, the practitioner will have at his disposal, on the one hand, a spatial and temporal map of break-ups of the tear film on the corneal surface and, on the other hand, a time-dependent curve tracing the appearance of the break-ups of the tear film as a function of time. This time-dependent curve, and especially its slope, will reveal the rate of appearance of break-ups in the tear film. This allows the interpretation of the examination performed to be refined.

The invention is not limited to the examples described above, merely by way of example, but encompasses any variant, such as any other distribution or variation in the height of the lines that those skilled in the art are able to envision, within the scope of the claimed protection. In particular, as stated above, the lines of the pattern, which are parallel horizontal lines in the shown example, may be replaced by vertical lines, a rotation of the image for example allowing the image-processing means for detecting the deformations of the lines to be applied to this configuration without changing the direction thereof.

What is claimed is:

1. Device for detecting one or more break-ups of a tear film, comprising a backlit translucent plate equipped with a test chart that is positioned in front of at least one eye of a patient and that is provided with a pattern that is reflected from the eye of the patient, and at least one digital photographic camera connected to a computing system provided with means for processing and analyzing images, an objective of the camera pointing toward the eye of the patient in order to photograph a reflection of the pattern of the test chart from the the eye of the patient wherein the pattern is provided with a series of lines taking the form of an alternation of horizontal or vertical transparent lines and opaque lines backlit through the translucent plate to form on said eye light lines and dark lines and wherein said means for processing and analyzing images are configured to detect deformations of said light lines or dark lines reflected from the eye of the patient and to identify tear-film break-ups revealed by these deformations.

2. The device as claimed in claim 1, wherein the lines of the series of lines are parallel lines.

3. The device as claimed in claim 1, wherein the width of the lines increases from a median line of the test chart toward edges of the test chart.

4. The device as claimed in claim 1, wherein the test chart is provided with a cylindrical curvature generated using a vertical generatrix, so that it forms a portion of a cylinder and follows the curvature of the head of the patient.

5. The device as claimed in claim 1, wherein said means for processing and analyzing images comprise means for converting the image to grayscale.

6. The device as claimed in claim 1, wherein said means for processing and analyzing images comprise anisotropic band-pass filtering means.

7. The device as claimed in claim 1, wherein said means for processing and analyzing images comprise means for analyzing the image in successions of pixels perpendicular to the direction of said lines, means for searching for bright or dark segments in said successions of pixels, and means for quantifying the size of said bright or dark segments and means for removing segments the size of which is incompatible with an image of thc lines of said series of lines of the pattern reflected on the eye of the patient.

8. The device as claimed in claim 7, wherein said means for processing and analyzing images comprise means for marking/cataloging bright or dark segment of objects, which are suitable for reconstructing first objects corresponding to lengths of light lines or dark lines reflected on the eye of the patient and for removing second objects of a shape incompatible with said light lines or dark lines.

9. The device as claimed in claim 8, wherein said means for processing and analyzing images comprise computing means for joining lengths of light or dark lines on the same axis, means for computing a polynomial regression on light-line data or dark-line data so as to compute RMS curves representative of edges of said lines and computing means for detecting regions of break-up of the tear film for image points of the line edges the distance of which to said curve is larger than a given tolerance value.

10. The device as claimed in claim 1, comprising means for tracking the eye of the patient or eyes of the patient based on iris recognition and tracking so as to align the detected tear-film break-ups with the analyzed eye or analysed eyes.

11. The device as claimed in claim 1, wherein the backlit translucent plate bearing the test chart and the one or more cameras are integrated into an ophthalmic measuring apparatus.

12. A method for detecting tear-film break-ups by means of a device as claimed in claim 1 comprising detecting an eyelid blink that delivers a start time, and performing at least one sequence comprising successively capturing images and computing regions of break-up from the start time to the next eyelid blink.

13. The method for detecting tear-film break-ups as claimed in claim 12, comprising capturing an image every 0.2 to 0.5 seconds and preferably every 0.3 seconds.

14. The method for detecting tear-film break-ups as claimed in claim 12, comprising, for each captured image, a succession of processing and analyzing steps comprising
converting the image to grayscale;
filtering the converted image by means of an anisotropic band-pass filter in order to decrease vignetting and to increase the uniformity of the brightness of the image;
searching for bright or dark segments in the image column by column, a step of quantifying the size of said bright or dark segments and a step of removing segments the size of which is incompatible with a correspondence with lines of the pattern;
a step of marking/cataloging bright segments or dark segments of objects, of reconstructing first of said objects corresponding to objects forming lengths of lines of the pattern, and a step removing second of said objects of shape incompatible with said lines of the pattern;
a step of joining lengths of lines of the same level, and a step for computing a polynomial regression on the line data so as to compute an RMS curve of the line edges;
computing regions of break-up of the tear film by computing the distance of points of line edges to said curve, said regions of break-up corresponding to line edges the distance of which to said curve is larger than a given tolerance value.

15. The method for detecting tear-film break-ups as claimed in claim 12, comprising steps of tracking the patient's eye or eyes by means of an iris-tracking method, so as to reposition the regions of break-up of the tear film that are detected with respect to the analyzed eye of the patient.

16. The method for detecting tear-film break-ups as claimed in claim 15, wherein the eye-tracking steps comprise:
a first step of transforming the image via application of an anisotropic band-pass filter that is applied in the direction of the width of the eye, to produce pairs of rising, dark to light, and falling, light to dark, transitions along a horizontal axis of the eye;
a step of segmenting the image to find the pairs of rising and falling transitions, which form segments that are necessarily representative of bright regions in the image;
a step of image filtering, which removes light segments from the central region comprising the pattern and top and bottom regions of the image;
a step of taking into account light segments, the other regions of the image no longer being considered in this analysis, and computing a first time an RMS circle of the perimeter of the iris on the basis of the right ends of the light segments on the left of the image and of the left ends of the light segments on the right of the image;
a step of removing points that are too far from the RMS circle; and
as regards the remaining points, a new step of computing an RMS circle to follow the outline of the iris.

17. A computer program comprising instructions for implementing the method as claimed in claim 12 when this program is executed by a processor.

18. A computer-readable non-volatile storage medium on which is stored a program for implementing the method as claimed in claim 12 when this program is executed by a processor.

19. A test chart for implementation of the device as claimed in claim 1, characterized in that it is produced by means of a transparent polymer film provided with opaque lines that are printed or screen-printed on said film and that are separated by transparent lines, said film being designed to be positioned on a backlit translucent plate of the device.

20. A test chart according to claim 19, comprising at least one empty region encircled by an opaque frame or at least one transparent substrate region in a median region of the test chart.
